(19) United States
(12) Patent Application Publication
(10) Pub. No.: US 2023/0298705 A1
(43) Pub. Date: Sep. 21, 2023

(54) Online Model Water Quality Conversion Method and System, Electronic Device, and Medium

(71) Applicant: BEIJING DRAINAGE GROUP CO., LTD, Beijing (CN)

(72) Inventors: Jiawei WANG, Beijing (CN); Xiaoyu MENG, Beijing (CN); Yong JIANG, Beijing (CN); Yang WEN, Beijing (CN); Hui ZHANG, Beijing (CN); Qun LI, Beijing (CN); Xing YUAN, Beijing (CN); Erlong JIAO, Beijing (CN); Yao LIU, Beijing (CN); Ye LI, Beijing (CN)

(21) Appl. No.: 18/004,253

(22) PCT Filed: Nov. 25, 2021
(86) PCT No.: PCT/CN2021/133140

§ 371 Date: Jan. 4, 2023

(30) Foreign Application Priority Data
Apr. 8, 2021 (CN) 202110377929.6

(51) Int. Cl. G16C 20/30; G16C 60/00; G16C 20/70; G06F 30/28

(52) U.S. Cl. CPC G16C 20/30; G16C 60/00; G16C 20/70; G06F 30/28

(57) ABSTRACT

An online model water quality conversion method and system, an electronic device, and a medium. The method may include: determining a type of online real-time data (101); establishing a conversion formula for calculation data and the online real-time data (102); acquiring water quality data over the years, determining conversion-related parameters of the conversion formula, and establishing a water quality data conversion model (103); and substituting the online real-time data obtained by real-time measurement into the water quality data conversion model, and performing real-time conversion to obtain the calculation data (104). According to the method, by converting online monitored data indexes into influent components required by the model, a sewage treatment plant model can be directly operated as an input source of an online simulation model, thereby laying a foundation for later simulation of effluent quality.

### FIELD OF TECHNOLOGY

The present disclosure relates to the field of online water quality data conversion simulation, and more particularly, to an online model water quality conversion method and system, an electronic device, and a medium.

### BACKGROUND

With the improvement of requirements on sewage treatment, during environmental monitoring, the effluent quality is required to reach the standard at all times. However, the biological treatment process of sewage is affected by fluctuation of an influent load and characteristics of a biological system itself, so it is very difficult to achieve standard operation at all times, and the safe and stable operation of a sewage treatment process has become a major challenge to the operation of sewage treatment plants. In the context, it has become an inevitable trend to change operation management from an extensive form based on experience to precise simulation and control depending on a model.

Initial activated sludge models began in the 1950s and 1960s, and the ASMs-series activated sludge models have been developed to be more mature. Process simulation (a mathematical model) has been widely accepted and applied in the design, upgrading and optimizing operation of municipal sewage treatment plants. There has been a strong theoretical foundation for the mathematical model which can simulate operation states of the sewage treatment plants. At present, existing commercial simulation software, such as BioWin, STOAT, and Aquasim has been widely applied, but these commercial software which has been developed to be more mature can only realize offline simulation of water plants, and cannot accurately simulate and control real-time states of the water plants on line. It is the biggest disadvantage of the current simulation technology, which will restrict the future development of the sewage treatment plants to the goal of “few-people on duty and even unmanned on duty”.

At present, an activated sludge process has been adopted in most sewage treatment plants in China, at the same time, online monitoring instruments can be disposed on water inlet ends of the water plants to monitor influent quality indexes, and therefore, adopting an online monitoring technology combined with ASMs series model suggested by the International Water Association (IWA) to perform real-time simulation and operation control on the water plants will become the future trend. However, it is difficult to convert data acquired by the online monitoring instruments into model water quality component data. Firstly, the influent components of the ASMs series model are relatively complex, and conventional influent quality indexes will be divided into a plurality of model influent components. With an ASM1 as an example, 13 influent components are involved, and how to convert the conventional influent quality indexes into the model influent components needs to be solved. Secondly, an influent component measurement method involved in the ASMs is tedious, many components cannot be directly measured by experiments, an influent component measurement method involved in offline simulation will hinder the online real-time simulation of the water plants, which becomes a major problem in intelligent control of the models. Thirdly, there are only online monitoring instruments for COD, ammonia nitrogen and pH of an influent in most sewage treatment plants which do not have the capability of detecting indexes such as SS and TN. For the situation, a set of systematic and scientific water quality conversion method for an ASM1 is to be provided.

Therefore, it is necessary to develop an online model water quality conversion method and system, an electronic device, and a medium.

The information disclosed in the background of the present disclosure is intended merely to deepen the understanding of the general background of the present disclosure, but should not be regarded as an admission or any form of suggestion that such information constitutes the prior art that is known to the skilled in the art.

### SUMMARY

The present disclosure provides an online model water quality conversion method and system, an electronic device, and a medium, according to which, by converting online monitored data indexes into influent components required by the model, a sewage treatment plant model can be directly operated as an input source of an online simulation model, thereby laying a foundation for later simulation of effluent quality.

In a first aspect, an embodiment of the present disclosure provides an online model water quality conversion method, including:
determining a type of online real-time data;
establishing a conversion formula for calculation data and the online real-time data;
acquiring water quality data over the years, determining conversion-related parameters of the conversion formula, and establishing a water quality data conversion model; and substituting the online real-time data obtained by real-time measurement into the water quality data conversion model, and performing real-time conversion to obtain the calculation data.

Preferably, the type of the online real-time data includes COD, ammonia nitrogen, and a pH value.

Preferably, the calculation data includes soluble inert organic matters, easily degradable organic matters, particulate inert organic matters, slowly degradable organic matters, heterotrophic bacteria, autotrophic bacteria, microbial decay products, dissolved oxygen, nitrate nitrogen, ammonia nitrogen, easily biodegradable organic nitrogen, slowly biodegradable organic nitrogen, and alkalinity.

Preferably, a conversion formula for each piece of calculation data and the online real-time data is established respectively, and then, a water quality data conversion formula corresponding to each piece of calculation data is determined.

Preferably, the method further includes:
substituting the calculation data into an ASM1 water plant full-process simulation model to simulate effluent quality.

Preferably, the method further includes:
operating the water quality data conversion model according to the online real-time data, performing real-time conversion to obtain the calculation data, and storing the calculation data in an online real-time database:
the ASM1 water plant full-process simulation model calling the calculation data in the online real-time database to simulate the effluent quality of a water plant and being used for simulating the effluent quality of the water plant on line.

Preferably, the method further includes:
in a Python environment, establishing the water quality data conversion model and the ASM1 water plant full-process simulation model at the same time, directly substituting the calculation data obtained by conversion into the ASM1 water plant full-process simulation model, and outputting an effluent quality result of the water plant and storing it in an online server database.

As a specific implementation of an embodiment of the present disclosure, in a second aspect, an embodiment of the present disclosure further provides an online model water quality conversion system, including:
a type determination module configured to determine a type of online real-time data;
a conversion formula establishment module configured to establish a conversion formula for calculation data and the online real-time data;
a water quality data conversion model establishment module configured to acquire water quality data over the years, determine conversion-related parameters of the conversion formula, and establish a water quality data conversion model; and
a conversion module configured to substitute the online real-time data obtained by real-time measurement into the water quality data conversion model, and perform real-time conversion to obtain the calculation data.

Preferably, the type of the online real-time data includes COD, ammonia nitrogen, and a pH value.

Preferably, the calculation data includes soluble inert organic matters, easily degradable organic matters, particulate inert organic matters, slowly degradable organic matters, heterotrophic bacteria, autotrophic bacteria, microbial decay products, dissolved oxygen, nitrate nitrogen, ammonia nitrogen, easily biodegradable organic nitrogen, slowly biodegradable organic nitrogen, and alkalinity.

Preferably, a conversion formula for each piece of calculation data and the online real-time data is established respectively, and then, a water quality data conversion formula corresponding to each piece of calculation data is determined.

Preferably, the system further includes:
the calculation data is substituted into an ASM1 water plant full-process simulation model to simulate effluent quality.

Preferably, the system further includes:
the water quality data conversion model is operated according to the online real-time data, real-time conversion is performed to obtain the calculation data, and the calculation data is stored in an online real-time database;
the ASM1 water plant full-process simulation model calls the calculation data in the online real-time database to simulate the effluent quality of a water plant and is used for simulating the effluent quality of the water plant on line.

Preferably, the system further includes:
in a Python environment, the water quality data conversion model and the ASM1 water plant full-process simulation model are established at the same time, the calculation data obtained by conversion is directly substituted into the ASM1 water plant full-process simulation model, and an effluent quality result of the water plant is output and stored in an online server database.

In a third aspect, an embodiment of the present disclosure further provides an electronic device, wherein the electronic device includes;
a memory storing executable instructions; and
a processor, the processor executing the executable instructions in the memory to implement the online model water quality conversion method.

In a fourth aspect, an embodiment of the present disclosure further provides a computer-readable storage medium, wherein the computer-readable storage medium stores a computer program that, when being executed by a processor, implements the online model water quality conversion method.

The present disclosure has the beneficial effects that:
(1) by converting online monitored conventional index data into the influent components required by the model, the sewage treatment plant model can be directly operated to solve the problems of complex influent components of the ASMs series model and difficulty in using conventional influent indexes, and the conventional influent indexes can be converted into components of the ASM1 so as to be used for a water plant simulation model in the present disclosure;
(2) it is difficult to measure the influent components of the ASMs series model, and there is a large workload to measure the influent components every day; and the present disclosure can be separated from the existing commercial simulation software, promote the development of water plant online simulation, simulate the effluent condition of the water plant in real time, provide a strong basis for the water plant to optimize and control an operation strategy, and also save the manpower and material resources brought by detection and offline simulation; and
(3) online monitoring instruments for the influent of the water plant are incomplete in monitoring indexes and only have the capability of detecting COD and ammonia nitrogen on line; and in the present disclosure, the problem can be solved, water quality conversion of components of the ASM1 can be performed by using the only instruments for monitoring the COD, the ammonia nitrogen and the pH value to perform simulation, and thus, the cost and pressure of the water plant in instrument installation, maintenance and calibration are reduced.

The method and system provided by the present disclosure have other characteristics and advantages that will be apparent from or are set forth in detail in the accompanying drawings and the following detailed description, which are incorporated herein and which together serve to explain specific principles of the present disclosure.

### BRIEF DESCRIPTION OF THE DRAWINGS

The above and other objectives, characteristics and advantages of the present disclosure will become more apparent by describing exemplary embodiments thereof in detail with reference to the accompanying drawings, wherein the exemplary embodiments of the present disclosure, the same reference numerals generally refer to the same parts.

FIG. 1 shows a process diagram of steps of an online model water quality conversion method according to an embodiment of the present disclosure:

FIG. 2 shows a schematic diagram of nitrogen-containing component proportioning based on a COD component according to an embodiment of the present disclosure; and

FIG. 3 shows a block diagram of an online model water quality conversion system according to an embodiment of the present disclosure.

Description for reference numerals in the accompanying drawings:

201, type determination module; 202, conversion formula establishment module; 203, water quality data conversion model establishment module; 204, conversion module.

### DESCRIPTION OF THE EMBODIMENTS

Hereinafter, preferred embodiments of the present disclosure will be described in more detail. Although preferred embodiments of the present disclosure are described as below, it should be understood that the present disclosure may be embodied in various forms and should not be limited by the embodiments set forth herein.

The present disclosure provides an online model water quality conversion method, including:
a type determination module configured to determine a type of online real-time data;
a conversion formula establishment module configured to establish a conversion formula for calculation data and the online real-time data;
a water quality data conversion model establishment module configured to acquire water quality data over the years, determine conversion-related parameters of the conversion formula, and establish a water quality data conversion model; and
a conversion module configured to substitute the online real-time data obtained by real-time measurement into the water quality data conversion model, and perform real-time conversion to obtain the calculation data.

In an example, the type of the online real-time data includes COD, ammonia nitrogen, and a pH value.

In an example, the calculation data includes soluble inert organic matters, easily degradable organic matters, particulate inert organic matters, slowly degradable organic matters, heterotrophic bacteria, autotrophic bacteria, microbial decay products, dissolved oxygen, nitrate nitrogen, ammonia nitrogen, easily biodegradable organic nitrogen, slowly biodegradable organic nitrogen, and alkalinity.

In an example, a conversion formula for each piece of calculation data and the online real-time data is established respectively, and then, a water quality data conversion formula corresponding to each piece of calculation data is determined.

In an example, the method further includes:
the calculation data is substituted into an ASM1 water plant full-process simulation model to simulate effluent quality.

In an example, the method further includes:
the water quality data conversion model is operated according to the online real-time data, real-time conversion is performed to obtain the calculation data, and the calculation data is stored in an online real-time database;
the ASM1 water plant full-process simulation model calls the calculation data in the online real-time database to simulate the effluent quality of a water plant and is used for simulating the effluent quality of the water plant on line.

In an example, the method further includes:
in a Python environment, the water quality data conversion model and the ASM1 water plant full-process simulation model are established at the same time, the calculation data obtained by conversion is directly substituted into the ASM1 water plant full-process simulation model, and an effluent quality result of the water plant is output and stored in an online server database.

Specifically, the general influent monitoring indexes of a sewage plant include: COD, ammonia nitrogen, and a pH value. With an ASM1 as an example, influent components include: soluble inert organic matters S_{I}, easily degradable organic matters S_{S}, particulate inert organic matters X_{I}, slowly degradable organic matters X_{S}, heterotrophic bacteria X_{BH}, autotrophic bacteria X_{BA}, microbial decay products X_{P}, dissolved oxygen S_{O}, nitrate nitrogen S_{NO}, ammonia nitrogen S_{NH}, easily biodegradable organic nitrogen SD, slowly biodegradable organic nitrogen X_{ND}, and alkalinity S_{ALK}.

The total COD in sewage can be expressed by the following formula:

COD_{T}=S_{S}+X_{S}+X_{BA}+X+S_{I}+X_{I} (1)

The total nitrogen in sewage can be expressed by the following formula:

TN=S_{NH}+S_{NO}+N_{org} (2)

Historical data of influent (COD, BOD₅, total nitrogen, ammonia nitrogen, and COD in influent obtained after flocculation and filtration) and secondary sedimentation effluent (soluble COD and soluble BOD₅) from a water plant daily or hourly or minutely is acquired.

Coefficients of a conversion relationship are obtained from the COD and the BOD₅ in the influent over the years by using a mathematical method; proportional coefficients of the easily degradable organic matters S_{S} and the soluble inert organic matters S_{I} to the COD in the influent are derived from historical detection data of soluble COD, soluble BOD₅ and the COD in the influent obtained after flocculation and filtration in the secondary sedimentation effluent; coefficients of a conversion relationship are obtained from historical data of total nitrogen and ammonia nitrogen in the influent by using a mathematical method; and related coefficients of nitrogen-containing components are obtained on the basis of the division of ingredients or components. The above-mentioned related parameters of the water quality data conversion model are water plant characteristic parameters which need to be determined according to the actual situation of each water plant.

A calculation formula for each component is established by combining intermediate state indexes, related parameters of water quality conversion and an empirical relationship with real-time data of online monitoring instruments.

According to the above-mentioned related parameters of the water quality data conversion model and a formula for a conversion relationship between online monitored data and the model influent components, a water quality data conversion model program is established by using Python. The water quality data conversion model established by using Python includes model input, a conversion relationship, and model output. Through codes, online real-time data input, model operation and conversion result output to a database are achieved.

By screening a point table (online influent data) related to the present disclosure in the database, performing real-time grabbing, storing the data to an online real-time data list, inputting the data into the water quality conversion model program in a form of direct transmission in the database or file transmission after data cleaning, converting online conventional water quality index data and outputting the same as the real-time model component data for subsequent use of an ASM1 water plant full-process simulation model, online simulation of the effluent quality of a sewage treatment plant is achieved.

According to the ASM1 water plant full-process simulation model and the calculation data, the online simulation of the effluent quality of the sewage treatment plant is achieved.

There are two solutions of using storage and using the water quality conversion model and the ASM1 water plant full-process simulation model:
(1) the water quality data conversion model is operated according to online real-time influent data, real-time model components of the water plant are output by the model and are stored in an online real-time database, and the model is used as an input source of the ASM1 water plant full-process simulation model; and the ASM1 water plant full-process simulation model calls real-time influent component data obtained from the conversion model in the database, and the water plant simulation model is operated and is used for simulating the effluent quality of the water plant on line.
(2) In the Python environment, the water quality data conversion model and the ASM1 water plant full-process simulation model are established at the same time, the calculation data obtained by conversion is directly substituted into the ASM1 water plant full-process simulation model, and an effluent quality result of the water plant is output and stored in an online server database.

In the present disclosure, the ASM1 is taken as an example, but the method can also be extended and applied to water quality conversion of various other activated sludge models such as ASM2. ASM2d and ASM3. At the same time, in the present disclosure, the water quality conversion method and model program established by only using the fewest online monitoring instruments (COD, ammonia nitrogen, and pH) can be applied to the simulation of a water plant with fewer online monitoring instruments. The method is also applicable when the online monitoring instruments are increased in types such as online monitoring instruments for nitrate nitrogen, BOD₃, etc., and related coefficients and formulas for the conversion relationship can be decreased accordingly.

In the present disclosure, the water quality conversion method is implemented by adopting a Python programming language, and a computer programming code for implementing the present disclosure may also be compiled by using one or more other languages or combinations thereof, such as Java, C++, and Matlab.

With regard to online data transmission, a database server transmission mode is used in the present disclosure, and data can also be transmitted through a network or a cloud platform. Furthermore, the model program can be deployed in a local application server, and can also be deployed in a remote computer or cloud platform.

The present disclosure further provides an online model water quality conversion system, including:
a type determination module configured to determine a type of online real-time data;
a conversion formula establishment module configured to establish a conversion formula for calculation data and the online real-time data;
a water quality data conversion model establishment module configured to acquire water quality data over the years, determine conversion-related parameters of the conversion formula, and establish a water quality data conversion model; and
a conversion module configured to substitute the online real-time data obtained by real-time measurement into the water quality data conversion model, and perform real-time conversion to obtain the calculation data.

In an example, the type of the online real-time data includes COD, ammonia nitrogen, and a pH value.

In an example, the calculation data includes soluble inert organic matters, easily degradable organic matters, particulate inert organic matters, slowly degradable organic matters, heterotrophic bacteria, autotrophic bacteria, microbial decay products, dissolved oxygen, nitrate nitrogen, ammonia nitrogen, easily biodegradable organic nitrogen, slowly biodegradable organic nitrogen, and alkalinity.

In an example, a conversion formula for each piece of calculation data and the online real-time data is established respectively, and then, a water quality data conversion formula corresponding to each piece of calculation data is determined.

In an example, the system further includes:
the calculation data is substituted into an ASM1 water plant full-process simulation model to simulate effluent quality.

In an example, the system further includes:
the water quality data conversion model is operated according to the online real-time data, real-time conversion is performed to obtain the calculation data, and the calculation data is stored in an online real-time database,
the ASM1 water plant full-process simulation model calls the calculation data in the online real-time database to simulate the effluent quality of a water plant and is used for simulating the effluent quality of the water plant on line.

In an example, the system further includes;
in a Python environment, the water quality data conversion model and the ASM1 water plant full-process simulation model are established at the same time, the calculation data obtained by conversion is directly substituted into the ASM1 water plant full-process simulation model, and an effluent quality result of the water plant is output and stored in an online server database.

Specifically, the general influent monitoring indexes of a sewage plant include: COD, ammonia nitrogen, and a pH value. With an ASM1 as an example, influent components include: soluble inert organic matters S_{I}, easily degradable organic matters S_{S}, particulate inert organic matters X_{I}, slowly degradable organic matters X_{S}, heterotrophic bacteria X_{BH}, autotrophic bacteria X_{BA}, microbial decay products X_{P}, dissolved oxygen S_{O}, nitrate nitrogen S_{NO}, ammonia nitrogen S_{NH}, easily biodegradable organic nitrogen S_{ND}, slowly biodegradable organic nitrogen X_{ND}, and alkalinity S_{ALK}.

The total COD in sewage is expressed by the formula (1), and the total nitrogen in sewage is expressed by the following formula (2).

Historical data of influent (COD, BOD₅, total nitrogen, ammonia nitrogen, and COD in influent obtained after flocculation and filtration) and secondary sedimentation effluent (soluble COD and soluble BOD₅) from a water plant daily or hourly or minutely is acquired.

Coefficients of a conversion relationship between the COD and the BOD₅ are obtained from the COD and the BOD₅ in the influent over the years by using a mathematical method; proportional coefficients of the easily degradable organic matters S_{S} and the soluble inert organic matters S_{I} to the COD in the influent are derived from historical detection data of soluble COD, soluble BOD₅ and the COD in the influent obtained after flocculation and filtration in the secondary sedimentation effluent; coefficients of a conversion relationship are obtained from historical detection data of total nitrogen and ammonia nitrogen in the influent by using a mathematical method; and related coefficients of nitrogen-containing components are obtained on the basis of the division of ingredients or components. The above-mentioned related parameters of the water quality data conversion model are water plant characteristic parameters which need to be determined according to the actual situation of each water plant.

A calculation formula for each component is established by combining intermediate state indexes, related parameters of water quality conversion and an empirical relationship with real-time data of online monitoring instruments.

According to the above-mentioned related parameters of the water quality data conversion model and a formula for a conversion relationship between online monitored data and the model influent components, a water quality data conversion model program is established by using Python. The model adopts the water quality data conversion model established by using Python and includes model input, a conversion relationship, and model output. Through codes, online real-time data input, model operation and conversion result output to a database are achieved.

By screening a point table (online influent data) related to the present disclosure in the database, performing real-time grabbing, storing the data to an online real-time data list, inputting the data into the water quality conversion model program in a form of direct transmission in the database or file transmission after data cleaning, converting online conventional water quality index data and outputting the same as the real-time model component data for subsequent use of an ASM1 water plant full-process simulation model, online simulation of the effluent quality of a sewage treatment plant is achieved.

According to the ASM1 water plant full-process simulation model and the calculation data, the online simulation of the effluent quality of the sewage treatment plant is achieved.

There are two solutions of using storage and using the water quality conversion model and the ASM1 water plant full-process simulation model:
(1) the water quality data conversion model is operated according to online real-time influent data, real-time model components of the water plant are output by the model and are stored in an online real-time database, and the model is used as an input source of the ASM1 water plant full-process simulation model; and the ASM1 water plant full-process simulation model calls real-time influent component data obtained from the conversion model in the database, and the water plant simulation model is operated and is used for simulating the effluent quality of the water plant on line. The water quality data conversion model and the ASM1 water plant full-process simulation model belong to two modules. If the water plant needs to be changed, only the water quality data conversion model needs to be transferred to a new water plant and debugged (modify the conversion-related parameters).
(2) In the Python environment, the water quality data conversion model and the ASM1 water plant full-process simulation model are established at the same time, the calculation data obtained by conversion is directly substituted into the ASM1 water plant full-process simulation model, and an effluent quality result of the water plant is output and stored in an online server database.

The present disclosure further provides an electronic device, wherein the electronic device includes: a memory storing executable instructions; and a processor, the processor executing the executable instructions in the memory to implement the above-mentioned online model water quality conversion method.

The present disclosure further provides a computer-readable storage medium, wherein the computer-readable storage medium stores a computer program that, when being executed by a processor, implements the above-mentioned online model water quality conversion method.

In order to facilitate understanding the solutions of the embodiments of the present disclosure and their effects, four specific application examples will be set forth below. It will be understood by those skilled in the art that the example is provided merely to facilitate understanding the present disclosure and any specific details thereof are not intended to limit the present disclosure in any way.

### Embodiment 1

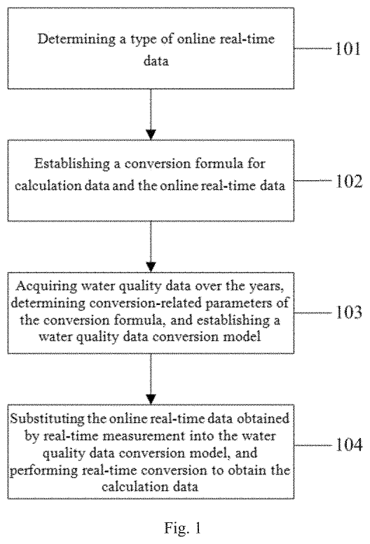

FIG. 1 shows a process diagram of steps of an online model water quality conversion method according to an embodiment of the present disclosure.

As shown in FIG. 1, the online model water quality conversion method includes: step 101, a type of online real-time data is determined; step 102, a conversion formula for calculation data and the online real-time data is established; step 103, water quality data over the years is acquired, conversion-related parameters of the conversion formula are determined, and a water quality data conversion model is established; and step 104, the online real-time data obtained by real-time measurement is substituted into the water quality data conversion model, and real-time conversion is performed to obtain the calculation data.

In step 101, the type of the online real-time data is determined to include COD, ammonia nitrogen, and a pH value.

In step 102, a conversion formula for each piece of calculation data and the online real-time data is established respectively, there are only online monitoring instruments for the COD, the ammonia nitrogen, and the pH value in a common sewage treatment plant, and a water quality data conversion formula is established according to the data of fewer type of online monitoring instruments.

(1) Water Quality Data Conversion Formulas Related to Online COD Instrument Data

If final model component calculation data is obtained, it is necessary to obtain the intermediate state indexes, five-day biochemical oxygen demand BOD₅, total biochemical oxygen demand BODᵤ as well as biodegradable COD (COD_{B}) and non-biodegradable COD (COD_{I}) in the influent.

There is a certain correlation between the COD and the BOD₅ in the influent, the two water quality indexes are in a one-dimensional linear relationship, and a relational formula can be obtained:

BOD₅=a·COD_{T,online}+b (3)

The measurement time of the BODᵤ is long, the BOD₅ is generally used as a conventional index for a water plant, but the BODᵤ can be derived from the BOD₅. In general domestic sewage, the BOD₅ is about 70% of the BODᵤ, and therefore, a relational formula can be obtained:

$${BOD}_{u} = \frac{B O {D}_{5}}{0.7} \qquad ( 4 )$$

For the determination of the COD_{B}, there are the following methods: (1) the BODᵤ in the sewage is totally regarded as the COD_{B}, this method is used in Canadian commercial simulation software GPS-X, that is, COD_{B}=BODᵤ; and according to the verification of a method experiment in Dutch wastewater characteristic guide, the following relational formula between BOD₂₀ and COD_{B} can be obtained:

COD_{B}=BOD₂₀=BODᵤ (5)

The total COD in the influent is composed of the COD_{B} and the COD_{I}, and therefore, the content of the COD_{I} can be obtained according to the data of an online monitoring instrument and the COD_{B} calculated as above:

COD_{I}=COD_{T,online}−COD_{B} (6)

In the above-mentioned formula, a and b are conversion coefficients of the COD and the BOD₅ (acquired in step 103), and COD_{T,online} is real-time COD data detected by an online monitoring instrument.

Thus, the easily degradable organic matters S_{S}, the soluble inert organic matters S_{I}, the heterotrophic bacteria X_{BH}, the autotrophic bacteria X_{BA}, the slowly degradable organic matters X_{S} and the particulate inert organic matters X_{I} as the COD components in the influent can be obtained:

S_{I}=S_{I}%·COD_{T,online} (7)

S_{S}=S_{S}%·COD_{T,online} (8)

X_{S}=COD_{B}−S_{S} (9)

X_{I}=COD_{I}−S_{I} (10)

X_{BA}=0 (11)

X_{BH}=0 (12)

wherein S_{I}% and S_{S}% are proportional coefficients of all of the above-mentioned components to the COD in the influent (acquired in step 103), and COD_{T,online} are real-time COD data detected by the online monitoring instruments. In general, there is an assumption in activated sludge models; the concentration of microorganisms in the influent is negligible as comparison with the amount of microorganisms generated in the process, and therefore, X_{BA} and X_{BH} are both 0.

(2) Water Quality Data Conversion Formulas related to Online Ammonia Nitrogen Instrument Data

There is a certain correlation between TN and NH₄-N in the influent, a large number of studies show that the two water quality indexes are in a one-dimensional linear relationship, and a formula can be obtained:

TN=c·NH4Nₒₙₗᵢₙₑ+d (13)

wherein c and d are conversion coefficients of ammonia nitrogen and total nitrogen (acquired in step 103).

The contents of the total nitrogen, the readily biodegradable organic nitrogen S_{ND}, the slowly biodegradable organic nitrogen X_{ND}, the nitrate nitrogen S_{NO} and the ammonia nitrogen S_{NH} in the influent which are related to nitrogen are obtained according to the online monitored ammonia nitrogen data of the influent, the conversion coefficients of the ammonia nitrogen and the total nitrogen and conversion coefficients of nitrogen contained in the COD components.

S_{ND}=i_{N,SS}·S_{S} (14)

X_{ND}=i_{N,XS}·X_{S} (15)

S_{NH}=NH4Nₒₙₗᵢₙₑ (16)

S_{NO}=TN−S_{NH}−S_{ND}−X_{ND}−i_{N,SI}·S_{I}−i_{N,XBH}·X_{BH}−i_{N,XI}·X_{I} (17)

wherein i_{N,SI}, i_{N,SS}, i_{N,XS}, i_{N,XBH}, i_{N,XI} are proportional coefficients of the nitrogen contained in each COD component (acquired in step 103), and NH4Nₒₙₗᵢₙₑ is real-time ammonia nitrogen data detected by an online monitoring instrument.

(3) Water Quality Data Conversion Formulas Related to Online pH Instrument Data

There is alkalinity (S_{ALK}) exists in the components of the ASM1, and the components can be converted from pH measurement values according to a conversion relationship between pH and alkalinity:

S_{ALK}=0.005005·10^{(pH}^{ₒₙₗᵢₙₑ}⁻⁷⁾ (18)

wherein pHₒₙₗᵢₙₑ is real-time pH data detected by an online monitoring instrument.

Step 103, water quality data over the years is acquired, conversion-related parameters of the conversion formula are determined, and a water quality data conversion model is established.

The water quality data over the years specifically includes COD. BOD₅, total nitrogen, ammonia nitrogen, and COD in influent obtained after flocculation and filtration, secondary sedimentation effluent data includes soluble COD and soluble BOD₅, and the historical data may be acquired daily or hourly or minutely.

(1) There is a certain correlation between the COD and the BOD₅ in the influent, and the two water quality indexes are in a one-dimensional linear relationship. According to the data of the COD and the BOD₅ in the influent over the years, linear equations for the COD and the BOD₅ are obtained through a least square method, and relational coefficients a and b are determined. The above-mentioned coefficients are related to actual influent characteristics of a water plant so as to be required to be determined according to the actual situation of each water plant.

(2) The proportional coefficients of the easily degradable organic matters S_{S} and the soluble inert organic matters S_{I} to the COD in the influent are derived from detection data of historical soluble COD, soluble BOD₅ and the COD in the influent obtained after flocculation and filtration in the secondary sedimentation effluent.

The content of the soluble inert organic matters S_{I} in the influent is approximately equal to the content of the soluble COD in the secondary sedimentation effluent. It is shown by the Dutch wastewater characteristics guide that the content of the soluble inert organic matters S_{I} can be derived from the soluble COD and the soluble BOD in the historical secondary sedimentation effluent, a ratio of the soluble inert organic matters to the total COD in the influent can be estimated, and then, the real-time content of the soluble inert organic matters S_{I} in the influent can be obtained. The related formulas are shown as follows.
\textcircled{1} a low-load sewage treatment plant:

$${S}_{I} = 0.9 \cdot {SCOD}_{out} \qquad ( 19 )$$

$${S}_{I} \% = \frac{0.9 \cdot {SCOD}_{out}}{{COD}_{T}} \qquad ( 20 )$$

\textcircled{2} a high-load sewage treatment plant:

$${S}_{I} = 0.9 \cdot {SCOD}_{out} - 1.5 \cdot {SBOD}_{5 , out} \qquad ( 21 )$$

$${S}_{I} \% = \frac{0.9 \cdot {SCOD}_{out} - 1.5 \cdot {SBOD}_{5 , out}}{{COD}_{T}} \qquad ( 22 )$$

In the formulas, SCODₒᵤₜ is the soluble COD in the effluent from a secondary sedimentation tank; SBOD_{5,out} is soluble BOD₅ in the secondary sedimentation effluent; and COD_{T} is the total COD in the influent.

The proportion of the easily degradable organic matters S_{S} can be calculated by the following formula:

$${S}_{S} \% = \frac{{COD}_{in , f} - {S}_{I}}{{COD}_{T}} \qquad ( 23 )$$

In the formula, COD_{in,f} is COD in the influent obtained after flocculation and filtration; S_{I} is the content of the soluble inert organic matters S_{I} calculated according to the above-mentioned formula; and COD_{T} is the total COD in the influent.

There is a certain correlation between the TN and NH₄-N in the influent, and a large number of studies show that the two water quality indexes are in a one-dimensional linear relationship. According to the historical detection data of the total nitrogen and the ammonia nitrogen in the influent, linear equations for the total nitrogen and the ammonia nitrogen are obtained through a least square method, and relational coefficients c and d are determined. The above-mentioned coefficients are related to actual influent characteristics of a water plant so as to be required to be determined according to the actual situation of each water plant.

(4) For the determination of the nitrogen-containing components, there are the two different ways: firstly, based on the division of ingredients, it is necessary to directly measure TiN, ammonia nitrogen and organic nitrogen in the influent, calibration parameters i_{XB} and i_{XP} in this part and the parameters in the model need to be repeatedly calibrated, and therefore, this method is feasible, but is complicated in operation and is risky; and secondly, based on the division of components, the content of the nitrogen component is simulated by the proportion of the COD component, this method is little in risk and easy to implement. In the present embodiment, the second way is adopted, i.e., the nitrogen-containing component is determined on the basis of the proportion of the COD component.

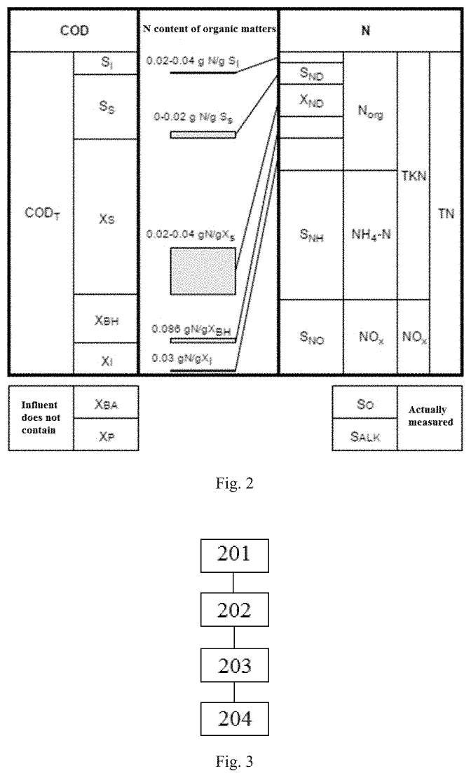

FIG. 2 shows a schematic diagram of nitrogen-containing component proportioning based on a COD component according to an embodiment of the present disclosure.

As shown in FIG. 2, according to the conventional nitrogen content of the COD component, conversion coefficients of the COD component and the N component are determined, wherein the value of i_{N,SI} ranges from 0.02 to 0.04, the value of i_{N,SS} ranges from 0 to 0.02, the value of i_{N,XS} ranges from 0.02 to 0.04, the value of i_{N,XBH} is 0.086, and the value of i_{N,XI} is 0.03.

The above-mentioned related parameters, obtained from the historical data, of the water quality data conversion model are water plant characteristic parameters which need to be determined according to the actual situation of each water plant, and coefficients of the nitrogen-containing components are nitrogen-containing proportions in all the COD components and may be standard values adopted by each plant.

According to the above-mentioned related parameters of the water quality data conversion model and a formula for a conversion relationship between online monitored data and the model influent components, a water quality data conversion model is established by using Python. Model input variables include: COD, ammonia nitrogen and pH data from the online monitoring instruments and related parameters of water plant characteristics; and model output variables include: 13 components in the influent of a water plant model based on an ASM1. The model adopts the water quality data conversion model established by using Python and includes model input, a conversion relationship, and model output. Through database transmission or excel files, online data input, model operation and conversion result output to a database are achieved.

Step 104, the online real-time data obtained by real-time measurement is substituted into the water quality data conversion model, and real-time conversion is performed to obtain the calculation data.

The data of the online monitoring instruments is stored in an Oracle database, and a point table related to the analysis of this project is screened out therefrom. In the present disclosure, data collection middleware HQVR is developed for COD, ammonia nitrogen and pH data serving as online monitored indexes, and the related data is driven through an oleDB interface, is grabbed in real time, is stored in a data list required by the present disclosure, and is input to a Python model to calculate real-time component data after the data is cleaned in a form of csv or excel files or a direct database transmission way so as to be used for an ASM1 water plant full-process simulation model.

With a reclaimed water plant in Beijing as an example, related coefficient values are obtained through water quality detection and data analysis. The specific coefficient values are shown in Table 1.

TABLE 1
Values in Examples of Water Quality
Conversion Related Coefficients

| | a | b | S_{I} % | S_{S} % |
|---|---|---|---|---|
| COD | 0.4085 | 20.2241 | 0.06 | 0.28 |

| | c | d | i_{N, SS} | i_{N, XS} | i_{N, SI} | i_{N, XBH} | i_{N, XI} |
|---|---|---|---|---|---|---|---|
| N | 0.9912 | 17.6643 | 0.01 | 0.03 | 0.03 | 0.086 | 0.03 |

Model components calculated according to the present disclosure are shown in PG-n Table 2.

TABLE 2

| | Day 1 | Day 2 | Day 3 | Day 4 | Day 5 |
|---|---|---|---|---|---|
| S₁ | 20.9 | 21.2 | 20.0 | 23.3 | 26.7 |
| S_{S} | 97.7 | 98.8 | 93.5 | 108.6 | 124.6 |
| X₁ | 95.5 | 96.9 | 90.2 | 109.4 | 129.7 |
| X_{S} | 134.8 | 136.1 | 130.3 | 146.7 | 164.0 |
| X_{BH} | 0 | 0 | 0 | 0 | 0 |
| X_{BA} | 0 | 0 | 0 | 0 | 0 |
| X_{P} | 0 | 0 | 0 | 0 | 0 |
| S_{O} | 0 | 0 | 0 | 0 | 0 |
| S_{NO} | 8.83 | 8.78 | 9.21 | 7.89 | 6.44 |
| S_{NH} | 36.10 | 31.00 | 34.60 | 34.70 | 41.10 |
| S_{ND} | 0.98 | 0.99 | 0.94 | 1.09 | 1.25 |
| X_{ND} | 4.05 | 4.08 | 3.91 | 4.40 | 4.92 |
| S_{ALK} | 0.02 | 0.01 | 0.01 | 0.01 | 0.02 |

Verification of Results in the Present Embodiment:

The results in the present embodiment are verified in terms of microbial concentration: since the amount of microorganisms in the conventional influent is negligible as comparison with the concentration of microorganisms in microbial biomass influent generated in the process, in the present embodiment, it is directly assumed that X_{BA} and X_{BH} are both 0. It is known by experience that the concentration of the autotrophic bacteria in the influent is 0, that is, X_{BA}=0; according to the content of the COD component, the X_{BH} is calculated according to a calculation formula shown as follows:

X_{BH}=COD_{T,online}−S_{S}−S₁−X_{S}−X_{I}−X_{BA} (24)

The content of the heterotrophic bacteria X_{BH} in the influent within 5 days is calculated to be 0 mg/l according to the above-mentioned relational formula of the COD component, which is the same as the assumption and meets assumed conditions, as shown in Table 3.

TABLE 3

| | Day 1 | Day 2 | Day 3 | Day 4 | Day 5 |
|---|---|---|---|---|---|
| Assumed Content of X_{BH} | 0 | 0 | 0 | 0 | 0 |
| Calculated Content of | 0 | 0 | 0 | 0 | 0 |
| X_{BH} Component | | | | | |

The results in the present embodiment are verified in terms of the classical value range of the mass fraction of each component: for municipal sewage, the proportion of each component in the influent is generally within a certain limited value range. Compared with the classical value range, calculation results in this example are all within the value range, as shown in Table 4, which shows that the results in the resent disclosure are valid.

TABLE 4

| | Day 1 | Day 2 | Day 3 | Day 4 | Day 5 | Classical Range |
|---|---|---|---|---|---|---|
| SI | 6% | 6% | 6% | 6% | 6% | 5-10% |
| SS | 28% | 28% | 28% | 28% | 28% | 12-30% |
| XS | 38.64% | 38.54% | 39.01% | 37.80% | 36.85% | 30-60% |
| XBA | 0% | 0% | 0% | 0% | 0% | 0-1% |

The effects of specific examples, achieved by Python, of the water quality conversion method based on the online monitoring instruments for the COD, the ammonia nitrogen and the pH according to the specific embodiments in the present disclosure are effective, which shows that the present disclosure is scientific and effective.

### Embodiment 2

FIG. 3 shows a block diagram of an online model water quality conversion system according to an embodiment of the present disclosure.

As shown in FIG. 3, the online model water quality conversion system includes:
a type determination module 201 configured to determine a type of online real-time data;
a conversion formula establishment module 202 configured to establish a conversion formula for calculation data and the online real-time data;
a water quality data conversion model establishment module 203 configured to acquire water quality data over the years, determine conversion-related parameters of the conversion formula, and establish a water quality data conversion model; and
a conversion module 204 configured to substitute the online real-time data obtained by real-time measurement into the water quality data conversion model, and perform real-time conversion to obtain the calculation data.

As an optional solution, the type of the online real-time data includes COD, ammonia nitrogen, and a pH value.

As an optional solution, the calculation data includes soluble inert organic matters, easily degradable organic matters, particulate inert organic matters, slowly degradable organic matters, heterotrophic bacteria, autotrophic bacteria, microbial decay products, dissolved oxygen, nitrate nitrogen, ammonia nitrogen, easily biodegradable organic nitrogen, slowly biodegradable organic nitrogen, and alkalinity.

As an optional solution, a conversion formula for each piece of calculation data and the online real-time data is established respectively, and then, a water quality data conversion formula corresponding to each piece of calculation data is determined.

As an optional solution, the system further includes:
the calculation data is substituted into an ASM1 water plant full-process simulation model to simulate effluent quality.

As an optional solution, the system further includes:
the water quality data conversion model is operated according to the online real-time data, real-time conversion is performed to obtain the calculation data, and the calculation data is stored in an online real-time database;
the ASM1 water plant full-process simulation model calls the calculation data in the online real-time database to simulate the effluent quality of a water plant and is used for simulating the effluent quality of the water plant on line.

As an optional solution, the system further includes:
in a Python environment, the water quality data conversion model and the ASM1 water plant full-process simulation model are established at the same time, the calculation data obtained by conversion is directly substituted into the ASM1 water plant full-process simulation model, and an effluent quality result of the water plant is output and stored in an online server database.

### Embodiment 3

The present disclosure provides an electronic device, wherein the electronic device includes: a memory storing executable instructions; and a processor, the processor executing the executable instructions in the memory to implement the above-mentioned online model water quality conversion method.

The electronic device according to the embodiment of the present disclosure includes the memory and the processor.

The memory is configured to store non-transitory computer-readable instructions. Specifically, the memory may include one or more computer program products which may include various forms of computer-readable storage media, such as a volatile memory and/or non-volatile memory. The volatile memory may include, for example, a random access memory (RAM) and/or a cache memory (cache), etc. The non-volatile memory may include, for example, a read-only memory (ROM), a hard disk, and a flash memory.

The processor may be a central processing unit (CPU) or other form of processing unit having data processing capability and/or instruction execution capability, and may control other components in the electronic device to perform desired functions. In an embodiment of the present disclosure, the processor is configured to execute the computer-readable instructions stored in the memory.

It should be understood by those skilled in the art that in order to solve the technical problem that how to obtain a good user experience effect is not known, known structures such as a communication bus and an interface may also be included in the present embodiment, and these known structures should also be included in the protection scope of the present disclosure.

The detailed description of the present embodiment may refer to the corresponding description in each of the above-mentioned embodiments and will not be described again herein.

### Embodiment 4

The embodiment of the present disclosure provides a computer-readable storage medium, wherein the computer-readable storage medium stores a computer program that, when being executed by a processor, implements the online model water quality conversion method.

The computer-readable storage medium according to the embodiment of the present disclosure stores non-transitory computer-readable instructions. The non-transitory computer-readable instructions, when executed by a processor, perform all or parts of the steps of the method according to each of the above-mentioned embodiments of the present disclosure.

The above-mentioned computer-readable storage medium includes, but is not limited to: an optical storage medium (e.g. CD-ROM and DVD), a magneto-optical storage medium (e.g. MO), a magnetic storage medium (e.g. a magnetic tape or a removable hard disk), a medium with a built-in rewritable non-volatile memory (e.g. a memory card) and a medium with a built-in ROM (e.g. a ROM cassette).

It should be understood by those skilled in the art that the foregoing description of the embodiments of the present disclosure is merely for purposes of illustrating the beneficial effects of the embodiments of the present disclosure, but is not intended to limit the embodiments of the present disclosure to any of the examples presented.

All the embodiments of the present disclosure have been described as above, the foregoing description is illustrative, not exhaustive, and not limited to all the disclosed embodiments. Many modifications and variations will be apparent to those of ordinary skill in the art without departing from the scope and spirit of all the embodiments described.

1. An online model water quality conversion method, comprising:
determining a type of online real-time data;
establishing a conversion formula for calculation data and the online real-time data;
acquiring water quality data over the years, determining conversion-related parameters of the conversion formula, and establishing a water quality data conversion model; and
substituting the online real-time data obtained by real-time measurement into the water quality data conversion model, and performing real-time conversion to obtain the calculation data.

2. The online model water quality conversion method of claim 1, wherein the type of the online real-time data comprises COD, ammonia nitrogen, and a pH value.

3. The online model water quality conversion method of claim 2, wherein the calculation data comprises soluble inert organic matters, easily degradable organic matters, particulate inert organic matters, slowly degradable organic matters, heterotrophic bacteria, autotrophic bacteria, microbial decay products, dissolved oxygen, nitrate nitrogen, ammonia nitrogen, easily biodegradable organic nitrogen, slowly biodegradable organic nitrogen, and alkalinity.

4. The online model water quality conversion method of claim 3, wherein a conversion formula for each piece of calculation data and the online real-time data is established respectively, and then, a water quality data conversion formula corresponding to each piece of calculation data is determined.

5. The online model water quality conversion method of claim 1, further comprising:
substituting the calculation data into an ASM1 water plant full-process simulation model to simulate effluent quality.

6. The online model water quality conversion method of claim 5, further comprising:
operating the water quality data conversion model according to the online real-time data, performing real-time conversion to obtain the calculation data, and storing the calculation data in an online real-time database;
the ASM1 water plant full-process simulation model calling the calculation data in the online real-time database to simulate the effluent quality of a water plant and being used for simulating the effluent quality of the water plant on line.

7. The online model water quality conversion method of claim 5, further comprising:
in a Python environment, establishing the water quality data conversion model and the ASM1 water plant full-process simulation model at the same time, directly substituting the calculation data obtained by conversion into the ASM1 water plant full-process simulation model, and outputting an effluent quality result of the water plant and storing it in an online server database.

8. An online model water quality conversion system, comprising:
a type determination module configured to determine a type of online real-time data;
a conversion formula establishment module configured to establish a conversion formula for calculation data and the online real-time data;
a water quality data conversion model establishment module configured to acquire water quality data over the years, determine conversion-related parameters of the conversion formula, and establish a water quality data conversion model; and
a conversion module configured to substitute the online real-time data obtained by real-time measurement into the water quality data conversion model, and perform real-time conversion to obtain the calculation data.

9. An electronic device, wherein the electronic device comprises:
a memory storing executable instructions; and
a processor, the processor executing the executable instructions in the memory to implement the online model water quality conversion method of claim 1.

10. A computer-readable storage medium, wherein the computer-readable storage medium stores a computer program that, when being executed by a processor, implements the online model water quality conversion method of claim 1.
